SEQUENCE LISTING (58 sequences)

SEQ ID NO: 1 - DNA, 181 bp, synthetic construct
  source 1..181: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 2 - DNA, 589 bp, synthetic construct
  source 1..589: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 3 - DNA, 154 bp, synthetic construct
  source 1..154: mol_type = other DNA
tagttattaatagtaatcaattacggggtcattagttcatagccgggactttccaaaatgtcgtaacaactccgccccattgacgcaaatgggcggtaggcgtgtacggtgggaggtctatataagcagagctggtttagtgaaccgtcagatc

SEQ ID NO: 4 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 5 - DNA, 22 bp, synthetic construct
  source 1..22: mol_type = other DNA
ctgtttcattcatctcagcagc

SEQ ID NO: 6 - DNA, 22 bp, synthetic construct
  source 1..22: mol_type = other DNA
ctttcaaaggtgataatctggg

SEQ ID NO: 7 - DNA, 22 bp, synthetic construct
  source 1..22: mol_type = other DNA
gtttcattcatctcagcagcag

SEQ ID NO: 8 - DNA, 22 bp, synthetic construct
  source 1..22: mol_type = other DNA
ttcattcatctcagcagcagtg

SEQ ID NO: 9 - DNA, 22 bp, synthetic construct
  source 1..22: mol_type = other DNA
tttaataataattattacggta

SEQ ID NO: 10 - DNA, 22 bp, synthetic construct
  source 1..22: mol_type = other DNA
ttttaataataattattacggt

SEQ ID NO: 11 - DNA, 22 bp, synthetic construct
  source 1..22: mol_type = other DNA
ttaataataattattacggtaa

SEQ ID NO: 12 - DNA, 22 bp, synthetic construct
  source 1..22: mol_type = other DNA
taaatattactgatttctgtca

SEQ ID NO: 13 - DNA, 22 bp, synthetic construct
  source 1..22: mol_type = other DNA
ttattacggtaaaacatcttga

SEQ ID NO: 14 - DNA, 22 bp, synthetic construct
  source 1..22: mol_type = other DNA
ataaatattactgatttctgtc

SEQ ID NO: 15 - DNA, 22 bp, synthetic construct
  source 1..22: mol_type = other DNA
aatattactgatttctgtcagt

SEQ ID NO: 16 - DNA, 22 bp, synthetic construct
  source 1..22: mol_type = other DNA
aaatattactgatttctgtcag

SEQ ID NO: 17 - DNA, 22 bp, synthetic construct
  source 1..22: mol_type = other DNA
cttctactgtttcattcatctc

SEQ ID NO: 18 - DNA, 22 bp, synthetic construct
  source 1..22: mol_type = other DNA
tattactgatttctgtcagtat

SEQ ID NO: 19 - DNA, 22 bp, synthetic construct
  source 1..22: mol_type = other DNA
acttctactgtttcattcatct

SEQ ID NO: 20 - DNA, 22 bp, synthetic construct
  source 1..22: mol_type = other DNA
gacaagcagaaagtccttcagg

SEQ ID NO: 21 - DNA, 22 bp, synthetic construct
  source 1..22: mol_type = other DNA
tataaatattactgatttctgt

SEQ ID NO: 22 - DNA, 22 bp, synthetic construct
  source 1..22: mol_type = other DNA
agatgacttctactgtttcatt

SEQ ID NO: 23 - DNA, 22 bp, synthetic construct
  source 1..22: mol_type = other DNA
catttctgagatgacttctact

SEQ ID NO: 24 - DNA, 22 bp, synthetic construct
  source 1..22: mol_type = other DNA
ttctgagatgacttctactgtt

SEQ ID NO: 25 - DNA, 157 bp, synthetic construct
  source 1..157: mol_type = other DNA
tcgacttcttaacccaacagaaggctcgagaaggtatattgctgttgccagtgagcgactgctgagatgaatgaaacagtagtgaagccacagatgtactgtttcattcatctcagcagctgcctactggctcggacttcaaggggctagaattcga

SEQ ID NO: 26 - DNA, 123 bp, synthetic construct
  source 1..123: mol_type = other DNA
aacttgtttattgcagcttataatggttacaaataaagcaatagcatcacaaatttcacaaataaagcatttttttcactgcattctagttgtggtttgtccaaactcatcaatgtatcttac

SEQ ID NO: 27 - DNA, 1177 bp, synthetic construct
  source 1..1177: mol_type = other DNA
ctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 28 - DNA, 230 bp, synthetic construct
  source 1..230: mol_type = other DNA
ctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 29 - DNA, 726 bp, synthetic construct
  source 1..726: mol_type = other DNA
gatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttca

SEQ ID NO: 30 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 31 - DNA, 765 bp, synthetic construct
  source 1..765: mol_type = other DNA
gagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgcccgtgttcctgcccgccaagcct

SEQ ID NO: 32 - protein, 255 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKP

SEQ ID NO: 33 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 34 - protein, 45 aa, synthetic construct
TTTPAPRPPTPAPTIASQPLSLRPEACRPAAGGAVHTRGLDFACD

SEQ ID NO: 35 - DNA, 72 bp, synthetic construct
  source 1..72: mol_type = other DNA
atctccctttactgcacatctgggcgcccttggccgggacttgtggggtccttctcctgtcactggttatca

SEQ ID NO: 36 - protein, 24 aa, synthetic construct
IYIWAPLAGTCGVLLLSLVITLYC

SEQ ID NO: 37 - DNA, 123 bp, synthetic construct
  source 1..123: mol_type = other DNA
aggagcaagaggagcaggctgctgcacagcgactacatgaacatgacccccaggaggcccggccccaccaggaagcactaccagccctacgccccccccagggacttcgccgcctacaggagc

SEQ ID NO: 38 - protein, 41 aa, synthetic construct
RSKRSRLLHSDYMNMTPRRPGPTRKHYQPYAPPRDFAAYRS

SEQ ID NO: 39 - DNA, 114 bp, synthetic construct
  source 1..114: mol_type = other DNA
tgctggctgaccaagaagaagtacagcagcagcgtgcacgaccccaacggcgagtacatgttcatgagggccgtgaacaccgccaagaagagcaggctgaccgacgtgaccctg

SEQ ID NO: 40 - protein, 38 aa, synthetic construct
CWLTKKKYSSSVHDPNGEYMFMRAVNTAKKSRLTDVTL

SEQ ID NO: 41 - DNA, 111 bp, synthetic construct
  source 1..111: mol_type = other DNA
aggagggaccagaggctgccccccgacgcccacaagccccccggcggcggcagcttcaggacccccatccaggaggagcaggccgacgcccacagcaccctggccaagatc

SEQ ID NO: 42 - protein, 37 aa, synthetic construct
RRDQRLPPDAHKPPGGGSFRTPIQEEQADAHSTLAKI

SEQ ID NO: 43 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 44 - protein, 42 aa, synthetic construct
KRGRKKLLYIFKQPFMRPVQTTQEEDGCSCRFPEEEEGGCEL

SEQ ID NO: 45 - DNA, 72 bp, synthetic construct
  source 1..72: mol_type = other DNA
ctgtgcgcacgcccacgccgcagccccgcccaagaagatggcaaagtctacatcaacatgccaggcaggggc

SEQ ID NO: 46 - protein, 24 aa, synthetic construct
LCARPRRSPAQEDGKVYINMPGRG

SEQ ID NO: 47 - DNA, 156 bp, synthetic construct
  source 1..156: mol_type = other DNA
tacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaa

SEQ ID NO: 48 - protein, 52 aa, synthetic construct
YFLGRLVPRGRGAAEAATRKQRITETESPYQELQGQRSDVYSDLNTQRPYYK

SEQ ID NO: 49 - DNA, 360 bp, synthetic construct
  source 1..360: mol_type = other DNA
tggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattcc

SEQ ID NO: 50 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 51 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 52 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 1206 bp, synthetic construct
  source 1..1206: mol_type = other DNA
atgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgac

SEQ ID NO: 54 - protein, 402 aa, synthetic construct
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVD

SEQ ID NO: 55 - DNA, 474 bp, synthetic construct
  source 1..474: mol_type = other DNA
atgctgaccagcctgctgtgctggatggccctgtgcctgctgggcgccgaccacgccgacgcctgcccctacagcaaccccagcctgtgcagcggcggcggcggcagcgagctgcccacccagggcaccttcagcaacgtgagcaccaacgtgagccccgccaagcccaccaccaccgcctgcccctacagcaaccccagcctgtgcagcggcggcggcggcagccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaaccacaggaacaggaggagggtgtgcaagtgccccaggcccgtggtgtaa

SEQ ID NO: 56 - protein, 157 aa, synthetic construct
MLTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 57 - DNA, 20 bp, synthetic construct
  source 1..20: mol_type = other DNA
aacagaaggctcgagaaggt

SEQ ID NO: 58 - DNA, 20 bp, synthetic construct
  source 1..20: mol_type = other DNA
ccttgaagtccgagccagta

(19) United States
(12) Patent Application Publication
(10) Pub. No.: US 2026/0166069 A1
(43) Pub. Date: Jun. 18, 2026

(54) ATTENUATION OF CYTOKINE RELEASE SYNDROME IN IMMUNOTHERAPY

(71) Applicant: MICROCRISPR PVT LTD., Vapi (IN)

(72) Inventors: Aaman Anjum BILAKHIA, Vapi (IN); Nilay Mohanlal LAD, Vapi (IN); Dhaval Ashvinbhai KEDARIA, Astan, Bardoli, Gujarat (IN); Urvi Hrishiraj PANWAR, Valsad, Gujarat (IN); Jyotirmoi AICH, Vapi, Gujarat (IN); Jitendra Devendra VYAS, Vapi, Gujarat (IN); Areej AKHTAR, New Delhi (IN)

(73) Assignee: MICROCRISPR PVT LTD., Vapi (IN)

(21) Appl. No.: 19/122,354

(22) PCT Filed: Oct. 20, 2023
(86) PCT No.: PCT/IN2023/050971

§ 371 Date: Apr. 18, 2025

(30) Foreign Application Priority Data
Oct. 20, 2022 (IN) 202221053834
Oct. 20, 2022 (IN) 202221053836

(51) Int. Cl. A61K 31/7105; C07K 14/705; C12N 5/0783; C12N 5/10; C12N 7/00; C12N 15/113; C12N 15/62; C12N 15/86

(52) U.S. Cl. CPC A61K 31/7105; C07K 14/70503; C12N 5/0636; C12N 5/10; C12N 7/00; C12N 15/113; C12N 15/625; C12N 15/86; C07K 2317/53; C07K 2317/622; C07K 2319/03; C12N 2310/141; C12N 2310/531; C12N 2795/00043; C12N 2800/10; C12N 2830/34; C12N 2830/50

(57) ABSTRACT

A recombinant nucleic acid molecule is encoded at least by an ORF to lower a cytokine storm during immunotherapy. The recombinant nucleic acid molecule includes at least one hairpin loop structure and a first promoter. The hairpin loop structure regulates an amount of Granulocyte monocyte colony stimulating factor (GMCSF) cytokine during immunotherapy. The hairpin loop structure is formed by one or more short hairpin RNA sequences. The first promoter is disposed upstream of the at least one hairpin loop structure.

### PRIORITY

This complete specification claims the benefit from the Indian Provisional Application No. 202221053834 and 202221053836, filed on 20 Oct. 2022, the contents of which are incorporated by reference herein.

### FIELD OF THE INVENTION

The present disclosure relates to an artificially modified immune effector cell to reduce cytokine release syndrome. More specifically, the present disclosure relates to an artificially modified immune effector cell that reduces level of granulocyte monocyte colony stimulating factor (GMCSF) released during immunotherapy for cancer.

### BACKGROUND OF THE INVENTION

Cancer is a disease in which abnormal cells divide uncontrollably and destroy body tissue. Preferably, the natural immune cells of the body recognize an advanced tumor as either “hot” or “cold” depending on whether immune cells are present or absent at the tumor site respectively. In case of hot-tumors, the immune cells (including cytotoxic T lymphocytes or T-cells) although present in abundance at the tumor site, are not active due to the inhibitory signals created by tumor microenvironment. While in the case of cold-tumors it is the chemotactic signaling of T-cells that is inhibited.

To enable the immune cells to actively target cancerous cells, Chimeric Antigen Receptor (CAR) T-cell based immunotherapy has been introduced. CAR T-cell based immunotherapy is an adoptive cell therapy (ACT) in which immune cells are engineered (genetically manipulated) to specifically target and kill cancer cells. In other words, the CAR constructs of the CAR T-cells are designed to target specific Tumor-associated antigens (TAAs).

Conventional CAR T-cell based therapies target single tumor antigen which is effective only against B cell malignancies. Hence, conventional CAR T-cell based therapies have limited application for other blood cell related malignancies (low CD19), where “off-target effect” is a major concern.

Although most adverse events of CAR T-cell immunotherapy are tolerable and acceptable, CAR T-cell immunotherapy is not without significant side effects. Conventionally available latest CAR T-cell based immunotherapy (based on second generation CAR design) often displays serious systemic toxicities that include release of a high amount of cytokines. Cytokines are signaling molecules that regulate immune system function. The release of high amount of cytokines termed as cytokine release syndrome (CRS), causes immune-related complications, and/or neurotoxicity. Neurotoxicity includes symptoms such as confusion, delirium, and seizures which further leads to rapid release of cytokines into the central nervous system. In CRS, the immune system overreacts and releases a barrage of cytokines, leading to patients developing a “storm” of cytokines immediately after being administered the CAR T-cell based immunotherapy. The released cytokines cause fever, low blood pressure, and difficulty in breathing. Granulocyte monocyte colony stimulating factor (GMCSF), an upregulated cytokine during CRS, is one of the key pro-inflammatory molecules which contributes to CRS.

For instance, FIG. 1 depicts an immune response of conventional CAR T-cell. The conventional CAR T-cell induces release of high levels of cytokines like GMCSF, Interleukin 6 (IL6), etc. The released GMCSF recruits pro-inflammatory immune cells like monocytes, neutrophils, basophils and macrophages. These pro-inflammatory immune cells induce (up-regulation) release of high levels of pro-inflammatory cytokines like IL6, Interleukin 1 (IL1), Nitric oxide (NO), etc. These high levels of pro-inflammatory cytokines eventually lead to CRS.

Although genome engineering approaches to completely knockout genes involved in CRS have been used, they have their limitations. For instance, the complete knockout of the genes may have an unwanted effect on the survival of the CAR T-cells and may also induce off-target effects.

Further, due to the nature of CAR T-cells as living therapeutics, the engineered CAR T-cells may transform into cancerous cells due to uncontrolled proliferation of the CAR T-cells.

Hence, CRS along with inability to control proliferation of the engineered CAR T-cells constitute a major challenge in the treatment of cancer patients with CAR T-cell based immunotherapies. Additionally, patients infused with conventional CAR T-cells have been diagnosed with relapse (i.e., reoccurrence of cancerous cells) as the conventional CAR T-cells do not persist for a long period of time.

In light of the aforementioned discussion, there exists a need for a novel CAR T-cell based immunotherapy that overcomes the problems associated with conventional CAR T-cell based immunotherapies.

### SUMMARY OF THE INVENTION

Particular embodiments of the present disclosure are described herein below with reference to the accompanying drawings, however, it is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

In an exemplary embodiment, the present disclosure relates to a recombinant nucleic acid molecule encoded at least by an ORF to lower a cytokine storm during immunotherapy. The recombinant nucleic acid molecule includes at least one hairpin loop structure and a first promoter. The hairpin loop structure regulates an amount of Granulocyte monocyte colony stimulating factor (GMCSF) cytokine during immunotherapy. The hairpin loop structure is formed by one or more short hairpin RNA sequences. The first promoter is disposed upstream of the at least one hairpin loop structure.

In an exemplary embodiment, the present disclosure relates to a recombinant nucleic acid molecule encoded at least by an ORF to lower a cytokine storm during immunotherapy. The recombinant nucleic acid molecule includes at least one hairpin loop structure, a first promoter, one or more chimeric antigen receptor genes, a second promoter, and one or more long terminal repeats. The hairpin loop structure regulates an amount of Granulocyte monocyte colony stimulating factor (GMCSF) cytokine during immunotherapy. The hairpin loop structure is formed by at least two short hairpin RNA sequences and at least one microRNA30 sequence. The short hairpin RNA sequences form stems of the hairpin loop structure, and the microRNA30 sequence forms a loop of the hairpin loop structure. The first promoter is disposed upstream of the at least one hairpin loop structure. The one or more chimeric antigen receptor genes expresses one or more single-chain variable fragment domains, one or more hinge domains, one or more co-stimulatory domains, one or more signaling domains, one or more safety switch domains, and one or more transmembrane domains. The single-chain variable fragment domains are configured to bind one or more tumor associated antigens. The hinge domains are coupled to the one or more single-chain variable fragment domains. The signaling domains are coupled to the one or more co-stimulatory domains. The safety switch domains are coupled to the signaling domains. The transmembrane domains operationally couple the hinge domains to the co-stimulatory domains. The second promoter is disposed upstream of the chimeric antigen receptor genes. The long terminal repeats are disposed upstream of the first promoter and downstream of the chimeric antigen receptor genes.

In an exemplary embodiment, the present disclosure relates to a recombinant nucleic acid molecule encoded at least by an ORF to lower a cytokine storm during immunotherapy. The recombinant nucleic acid molecule includes at least one hairpin loop structure, a first promoter, a poly-A-tail sequence, one or more chimeric antigen receptor genes, a second promoter, and one or more long terminal repeats. The hairpin loop structure regulates an amount of Granulocyte monocyte colony stimulating factor (GMCSF) cytokine during immunotherapy. The hairpin loop structure is formed by at least two short hairpin RNA sequences and at least one microRNA30 sequence. The short hairpin RNA sequences form stems of the hairpin loop structure, and the microRNA30 sequence forms a loop of the hairpin loop structure. The first promoter is disposed upstream of the at least one hairpin loop structure. The poly-A-tail sequence is disposed downstream of the hairpin loop structure. The one or more chimeric antigen receptor genes expresses one or more single-chain variable fragment domains, one or more hinge domains, one or more co-stimulatory domains, one or more signaling domains, one or more safety switch domains, and one or more transmembrane domains. The single-chain variable fragment domains are configured to bind one or more tumor associated antigens. The hinge domains are coupled to the one or more single-chain variable fragment domains. The signaling domains are coupled to the one or more co-stimulatory domains. The safety switch domains are coupled to the signaling domains. The transmembrane domains operationally couple the hinge domains to the co-stimulatory domains. The second promoter is disposed upstream of the chimeric antigen receptor genes. The long terminal repeats are disposed upstream of the first promoter and downstream of the chimeric antigen receptor genes.

In an exemplary embodiment, the present disclosure relates to a recombinant nucleic acid molecule encoded at least by an ORF to lower a cytokine storm during immunotherapy. The recombinant nucleic acid molecule includes at least one hairpin loop structure, a first promoter, one or more chimeric antigen receptor genes, a second promoter, and one or more long terminal repeats. The hairpin loop structure regulates an amount of Granulocyte monocyte colony stimulating factor (GMCSF) cytokine during immunotherapy. The hairpin loop structure is formed by one or more short hairpin RNA sequences. The first promoter is disposed upstream of the at least one hairpin loop structure. The one or more chimeric antigen receptor genes expresses one or more single-chain variable fragment domains, one or more hinge domains, one or more co-stimulatory domains, one or more signaling domains, one or more safety switch domains, and one or more transmembrane domains. The single-chain variable fragment domains are configured to bind one or more tumor associated antigens. The hinge domains are coupled to the one or more single-chain variable fragment domains. The signaling domains are coupled to the one or more co-stimulatory domains. The safety switch domains are coupled to the signaling domains. The transmembrane domains operationally couple the hinge domains to the co-stimulatory domains. The second promoter is disposed upstream of the chimeric antigen receptor genes. The long terminal repeats are disposed upstream of the first promoter and downstream of the chimeric antigen receptor genes.

In an exemplary embodiment, the present disclosure relates to a transcript of a recombinant nucleic acid molecule encoded at least by an ORF to lower a cytokine storm during immunotherapy. The transcript includes a messenger RNA of a hairpin loop structure transcribed from the recombinant nucleic acid molecule. The messenger RNA of the hairpin loop structure is configured to bind with a messenger RNA of a cytokine.

In an exemplary embodiment, the present disclosure relates to a vector including at least one of a recombinant nucleic acid molecule ligated to at least one of a plasmid, a cosmid, a viral vector and a phage. The recombinant nucleic acid molecule encoded at least by an ORF to lower a cytokine storm during immunotherapy.

In an exemplary embodiment, the present disclosure relates to an engineered immune cell including at least one of a recombinant nucleic acid molecule encoded at least by an ORF to lower a cytokine storm during immunotherapy.

In an exemplary embodiment, the present disclosure relates to a composition including an engineered immune cell suspended in a nutrient medium. The engineered immune cell including at least one of a recombinant nucleic acid molecule encoded at least by an ORF to lower a cytokine storm during immunotherapy.

In an exemplary embodiment, the present disclosure relates to a method to prepare an engineered immune cell. The method includes isolating a plurality of T-cells from a population of Peripheral Blood Mononuclear Cells. Replicating at least one recombinant nucleic acid molecule, encoded at least by an ORF to lower a cytokine storm during immunotherapy, in a vector. And, delivering the vector within the plurality of T-cells.

The foregoing features and other features as well as the advantages of the invention will become more apparent from the following detailed description, which proceeds with reference to the accompanying figures. In the accompanying drawings, like reference numbers indicate identical or like parts throughout the several views. These features will be described at sufficiently detailed levels to allow the technicians of the subject matter to implement the invention. Also, it is understood that other features can be used and structural changes can be made without leaving the scope of the invention.

### BRIEF DESCRIPTION OF THE DRAWINGS

The summary above, as well as the following detailed description of illustrative embodiments, is better understood when read in conjunction with the apportioned drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale.

FIG. 1 depicts an illustration of CRS induction (Prior art), in accordance with one or more exemplary embodiments of the present disclosure.

FIG. 2 depicts an ORF 100 in accordance with one or more exemplary embodiments of the present disclosure.

FIG. 2a depicts an ORF 100a in accordance with one or more exemplary embodiments of the present disclosure.

FIG. 2b depicts an ORF 100b without a miR30 130a sequence in accordance with one or more exemplary embodiments of the present disclosure.

FIG. 3 depicts a CAR construct 200 in accordance with one or more exemplary embodiments of the present disclosure.

FIG. 3a depicts a CAR construct 300 in accordance with one or more exemplary embodiments of the present disclosure.

FIG. 4 depicts an illustration of CRS reduction in accordance with one or more exemplary embodiments of the present disclosure.

FIG. 5 depicts a method 400 to make CAR T-cell in accordance with one or more exemplary embodiments of the present disclosure.

FIGS. 6-13 depicts experimental observations in accordance with one or more exemplary embodiments of the present disclosure.

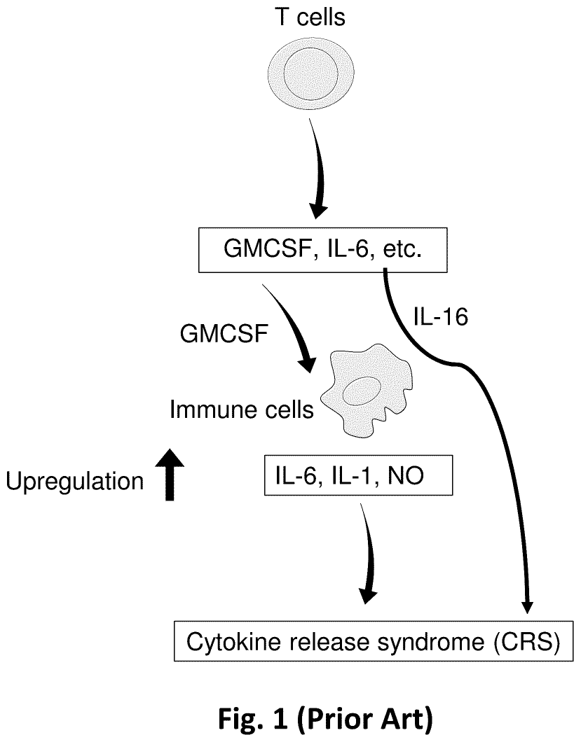

### DETAILED DESCRIPTION OF THE DRAWINGS

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like; Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

The term “activation,” as used herein, refers to the state of a cell that has been sufficiently stimulated to induce detectable cellular proliferation and/or differentiation into effector T-cells (or activated T-cells). Activation can also be associated with induced cytokine production and detectable effector functions.

The term “activated T cells” refers to, among other things, T cells (or CAR T-cells) that express the chimeric antigen receptor (CAR) construct and/or are able to bind to tumor associated antigens (TAAs).

The term “antibody” is used in the broadest sense and refers to monoclonal antibodies (including full length monoclonal antibodies), polyclonal antibodies, multi-specific antibodies (e.g., bispecific antibodies), and antibody fragments so long as they exhibit the desired biological activity or function. The antibodies in the present disclosure may exist in a variety of forms including, for example, polyclonal antibodies; monoclonal antibodies; Fv, Fab, Fab′, and F(ab′)2 fragments; as well as single chain antibodies and humanized antibodies.

The term “antibody fragments” refers to a portion of a full-length antibody, for example, the antigen binding or variable region of the antibody. Other examples of antibody fragments include Fab, Fab′, F(ab′)2, and Fv fragments; diabodies; linear antibodies; single-chain antibody molecules; and multi-specific antibodies formed from antibody fragments.

The term “Fv” refers to the minimum antibody fragment which contains a complete antigen-recognition and -binding site. This fragment consists of a dimer of one heavy- and one light-chain variable region domain in tight, non-covalent association. From the folding of these two domains emanates six hypervariable loops (3 loops each from the H and L chain) that contribute amino acid residues for antigen binding and confer antigen binding specificity to the antibody. However, even a single variable domain (or half of an Fv including only three complementarity determining regions (CDRs) specific for an antigen) has the ability to recognize and bind antigen, although at a lower affinity than the entire binding site (the dimer).

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. K and A light chains refer to the two major antibody light chain isotypes.

The term “synthetic antibody” refers to an antibody which is generated using recombinant DNA technology, such as, for example, an antibody expressed by a bacteriophage. The term also includes an antibody which has been generated by the synthesis of a DNA molecule encoding the antibody and the expression of the DNA molecule to obtain the antibody or to obtain an amino acid encoding the antibody. The synthetic DNA is obtained using technology that is available and well known in the art.

The term “antigen” refers to a molecule that provokes an immune response, which may involve either antibody production, or the activation of specific immunologically-competent cells, or both. Antigens include any macromolecule, including all proteins or peptides, or molecules derived from recombinant or genomic DNA. For example, DNA including a nucleotide sequence or a partial nucleotide sequence encoding a protein or peptide that elicits an immune response, and therefore, encodes an “antigen” as the term is used herein. An antigen need not be encoded solely by a full-length nucleotide sequence of a gene. An antigen can be generated, synthesized or derived from a biological sample including a tissue sample, a tumor sample, a cell, or a biological fluid.

The term “anti-tumor effect” as used herein, refers to a biological effect associated with a decrease in tumor volume, a decrease in the number of tumor cells, a decrease in the number of metastases, decrease in tumor cell proliferation, decrease in tumor cell survival, an increase in life expectancy of a subject having tumor cells, or amelioration of various physiological symptoms associated with the cancerous condition. An “anti-tumor effect” can also be manifested by the ability of the peptides, polynucleotides, cells, and antibodies in the prevention of the occurrence of tumor in the first place.

The term “auto-antigen” refers to an endogenous antigen mistakenly recognized by the immune system as being foreign. Auto-antigens include cellular proteins, phosphoproteins, cellular surface proteins, cellular lipids, nucleic acids, glycoproteins, including cell surface receptors.

The term “autologous” is used to describe a material derived from a subject which is subsequently re-introduced into the same subject.

The term “allogeneic” is used to describe a graft derived from a different subject of the same species. As an example, a donor subject may be a related or unrelated to the recipient subject, but the donor subject has immune system markers which are similar to the recipient subject.

The term “xenogeneic” is used to describe a graft derived from a subject of a different species. As an example, the donor subject is from a different species than a recipient subject, and the donor subject and the recipient subject can be genetically and immunologically incompatible.

The term “cancer” is used to refer to a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules, or there may be “complete” or “total” complementarity between the nucleic acids. The degree of complementarity between nucleic acid strands has significant effects on the efficiency and strength of hybridization between nucleic acid strands.

The term “corresponds to” or “corresponding to” refers to (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

The term “co-stimulatory ligand,” refers to a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including at least one of proliferation, activation, differentiation, and other cellular responses. A co-stimulatory ligand also includes, inter alia, an agonist or an antibody that specifically binds with a co-stimulatory molecule present on a T cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds CD83.

The term “co-stimulatory molecule” refers to the cognate binding partner on a T cell that specifically binds with a co-stimulatory ligand, thereby mediating a co-stimulatory response by the T cell, such as proliferation. Co-stimulatory molecules include an MHC class I molecule, BTLA, and a Toll-like receptor.

The term “co-stimulatory signal” refers to a signal, which in combination with a primary signal, such as TCR/CD3 ligation, leads to T cell proliferation and/or upregulation or downregulation of key molecules.

The term “encoding” refers to the inherent property of specific sequences of nucleotides in a polynucleotide, such as a gene, a cDNA, or an mRNA, to serve as a template for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

The term “exogenous” refers to a molecule that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides and proteins, the term “endogenous” or “native” refers to naturally occurring polynucleotide or amino acid sequences that may be found in a given wild-type cell or organism. Also, a particular polynucleotide sequence that is isolated from a first organism and transferred to a second organism by molecular biological techniques is typically considered an “exogenous” polynucleotide or amino acid sequence with respect to the second organism. In specific embodiments, polynucleotide sequences can be “introduced” by molecular biological techniques into a microorganism that already contains such a polynucleotide sequence, for instance, to create one or more additional copies of an otherwise naturally occurring polynucleotide sequence, and thereby facilitate overexpression of the encoded polypeptide.

The term “expression” refers to the transcription and/or translation of a particular nucleotide sequence driven by its promoter.

The term “expression vector” refers to a vector including a recombinant polynucleotide including expression control (regulatory) sequences operably linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

In the context of the present disclosure, the following abbreviations for the commonly occurring nucleic acid bases are used. “A” refers to adenine, “C” refers to cytosine, “G” refers to guanine, “T” refers to thymine, and “U” refers to uracil.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. Moreover, the use of lentiviruses enables integration of the genetic information into the host chromosome resulting in stably transduced genetic information. HIV, SIV, and FIV are all examples of lentiviruses. Vectors derived from lentiviruses offer the means to achieve significant levels of gene transfer in vivo.

The term “modulating,” refers to mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

The term “under transcriptional control” refers to a promoter being operably linked to and in the correct location and orientation in relation to a polynucleotide to control (regulate) the initiation of transcription by RNA polymerase and expression of the polynucleotide.

The term “overexpressed” tumor antigen or “overexpression” of the tumor antigen is intended to indicate an abnormal level of expression of the tumor antigen in a cell from a disease area such as a solid tumor within a specific tissue or organ of the patient relative to the level of expression in a normal cell from that tissue or organ. Patients having solid tumor, or a hematological malignancy characterized by overexpression of the tumor antigen can be determined by standard assays known in the art.

The terms “patient,” “subject,” and “individual,” and the like are used interchangeably herein and refer to any human, or animal, amenable to the methods described herein. In certain non-limiting embodiments, the patient, subject, or individual is a human or animal. In embodiments, the term “subject” is intended to include living organisms in which an immune response can be elicited (e.g., mammals). Examples of subjects include humans, and animals, such as dogs, cats, mice, rats, and transgenic species thereof.

A subject in need of treatment or in need thereof includes a subject having a disease, condition, or disorder that needs to be treated. A subject in need thereof also includes a subject that needs treatment for prevention of a disease, condition, or disorder.

The term “polynucleotide” or “nucleic acid” refers to mRNA, RNA, CRNA, rRNA, cDNA or DNA. The term typically refers to a polymeric form of nucleotides of at least 10 bases in length, either ribonucleotides or deoxynucleotides or a modified form of either type of nucleotide. The term includes all forms of nucleic acids including single and double-stranded forms of nucleic acids.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally occurring allelic variants and orthologs.

The terms “polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally occurring amino acid polymers. In certain aspects, polypeptides may include enzymatic polypeptides, or “enzymes,” which typically catalyze (i.e., increase the rate of) various chemical reactions.

The term “polypeptide variant” refers to polypeptides that are distinguished from a reference polypeptide sequence by the addition, deletion, or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted or replaced with different amino acid residues.

The term “promoter” refers to a DNA sequence recognized by the synthetic machinery of the cell or introduced synthetic machinery, required to initiate the specific transcription of a polynucleotide sequence. The term “expression control (regulatory) sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The term “bind,” “binds,” or “interacts with” refers to a molecule recognizing and adhering to a second molecule in a sample or organism but does not substantially recognize or adhere to other structurally unrelated molecules in the sample. The term “specifically binds,” as used herein with respect to an antibody, refers to an antibody which recognizes a specific antigen, but does not substantially recognize or bind other molecules in a sample. For example, an antibody that specifically binds an antigen from one species may also bind that antigen from one or more species. But, such cross-species reactivity does not itself alter the classification of an antibody as specific. In another example, an antibody that specifically binds an antigen may also bind different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds a specific protein structure rather than to any protein. If an antibody is specific for epitope “A,” the presence of a molecule containing epitope A (or free, unlabeled A), in a reaction containing labeled “A” and the antibody, will reduce the amount of labeled A bound to the antibody.

The term “stimulation,” refers to a primary response induced by binding of a stimulatory molecule (e.g., a TCR/CD3 complex) with its cognate ligand thereby mediating a signal transduction event, such as signal transduction via the TCR/CD3 complex. Stimulation can mediate altered expression of certain molecules, such as downregulation of TGF-β, and/or reorganization of cytoskeletal structures.

The term “stimulatory molecule” refers to a cognate stimulatory ligand present on an antigen presenting cell that specifically binds to a molecule on a T cell.

The term “stimulatory ligand” refers to a ligand that when present on an antigen presenting cell (e.g., an APC, a dendritic cell, a B-cell, and the like) can specifically bind with a cognate binding partner (referred to herein as a “stimulatory molecule”) on a cell, for example a T cell, thereby mediating a primary response by the T cell, including activation, initiation of an immune response, proliferation, and similar processes.

The term “transfected” or “transformed” or “transduced” refers to a process by which an exogenous nucleic acid is transferred or introduced into the host cell. A “transfected” or “transformed” or “transduced” cell is one which has been transfected, transformed, or transduced with exogenous nucleic acid. The cell includes the primary subject cell and its progeny.

A “chimeric antigen receptor” (CAR) molecule is a recombinant polypeptide including at least an extracellular domain, a transmembrane domain and a cytoplasmic domain or intracellular domain.

Although the operations of exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

The present disclosure discloses a Chimeric Antigen Receptor T-cell(s) (CAR T-cell). The CAR T-cell is artificially engineered by genetic manipulation. The CAR T-cells may be derived from either an autologous or an allogeneic source. The CAR T-cell may be used to target different cellular disorders including but not limited to B cell malignancy (blood cancer), solid tumors, etc. In an embodiment, the CAR T-cells are directed towards relapsed or refractory B-cell acute lymphoblastic leukemia (ALL) or non-Hodgkin lymphoma (NHL).

The CAR T-cells may be genetically engineered to include one or more sequences that express one or more chimeric antigen receptors (CARs or CAR constructs). The CARs have pre-defined specificity towards one or more tumor associated antigens (TAAs) including but not limited to Cluster of Differentiation 19 (CD19), Cluster of Differentiation 7 (CD7), Cluster of Differentiation 20 (CD20), Cluster of Differentiation 22 (CD22), Cluster of Differentiation 123 (CD123), Cluster of Differentiation 133 (CD133), Cluster of Differentiation 30 (CD30), Cluster of Differentiation 138 (CD138), Epidermal Growth Factor Receptor (EGFR), Epidermal Growth Factor Receptor Variant III (EGFRvIII), Fibroblast Activation Protein a (FAP), Mucin 1 (MUC1), Disialoganglioside GD2 (GD2), Carcinoembryonic Antigen (CEA), Prostate-specific Membrane Antigen (PSMA), Human Epidermal Growth Factor Receptor 2 (HER2), New York Esophageal Squamous Cell Carcinoma-1 (NY-ESO-1), Melanoma-associated Antigen 3 (MAGEA-A3), Human Telomerase Reverse Transcriptase (hTERT), etc. In an exemplary embodiment, the CAR T-cells include CARs specific to CD19.

Further, the CAR T-cells includes at least one short hairpin RNA (shRNA) sequence to lower a cytokine storm called cytokine release syndrome (CRS) during immunotherapy. During CRS, a rapid release of cytokines is observed. Granulocyte Monocyte Colony Stimulating Factor (or Granulocyte Macrophage Colony Stimulating Factor) (GMCSF/GM-CSF) is one of the cytokines that is released at the time of immunotherapy. The shRNA sequence may inhibit cytokine production and/or cytokine signaling via RNA interference (RNAi). In an exemplary embodiment, a transcript of the shRNA sequence binds with an mRNA for the cytokine to functionally inactivate the mRNA and/or degrade the mRNA. In another exemplary embodiment, the transcript of the shRNA sequence binds with the mRNA of the cytokine to prevent its translation.

In another exemplary embodiment, the CAR T-cells of the present disclosure produce a transcript of at least one of twenty shRNA sequences specific to Granulocyte Monocyte Colony Stimulating Factor (or GMCSF) that contributes to cytokine release syndrome (CRS) at the time of immunotherapy. In an exemplary embodiment, the shRNA sequence regulates (i.e., reduces) the amount of GMCSF produced by selectively binding to the GMCSF mRNA and rendering it non-functional. GMCSF is a critical molecule involved in eliciting CRS and leading to premature CAR T-cell apoptosis. By regulating the amount of GM-CSF produced, the CAR T-cells as disclosed herein have lower risk of CRS and persist longer thereby, having ability to remove the cancerous cells safely and effectively.

Further, the CARs include a safety switch (SS) domain for regulating proliferation of the CAR T-cells thereby inhibiting transformation of the CAR T-cells into cancerous cells (for example, during manufacturing or post-infusion of the CAR T-cells in the patient). The SS domain may include but not limited to inducible caspase 9 (iCaspase 9), truncated Epidermal growth factor receptor (EGFRt), RQR8 or a combination thereof.

Hence, owing to the above CAR T-cells as disclosed herein are programmed to elicit an artificial immune response against the cancerous cells without any undesirable side effects of CRS. Further, based on the conditions and requirements of a patient (or subject), the CAR T-cells of the present disclosure may be selectively depleted to prevent malignant transformation of the CAR T-cells.

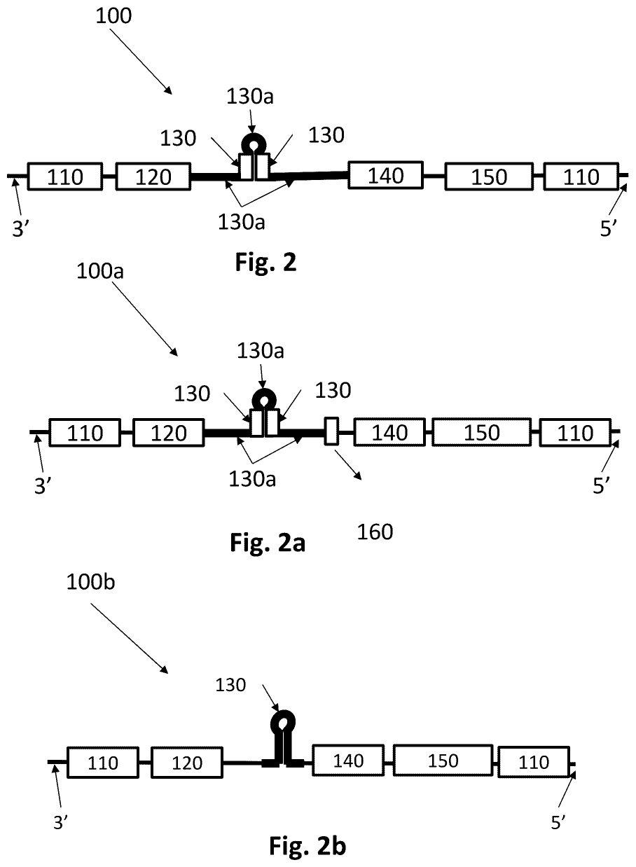

Now referring to the figures, FIG. 2 depicts an anti-sense (or template) strand of an open reading frame (ORF) 100 of a CAR T-cell (not shown). Accordingly, the ORF 100 may include a sense (or coding) strand (not shown) complementary to the anti-sense strand. The anti-sense strand of the ORF 100 extends from a 3′end to a 5′ end. Hence, in terms of direction, the 3′ end of the anti-sense strand of the ORF 100 is upstream and 5′ end is downstream. A recombinant nucleotide sequence molecule is at least encoded by the ORF 100.

At least a portion of the ORF 100 (or the recombinant nucleotide sequence molecule) may be introduced into one or more natural immune cells via genetic manipulation to produce an engineered immune cell. The ORF 100 (or a portion thereof) expresses one or more CARs (i.e., a protein and/or a polypeptide) and/or one or more shRNA transcripts (described below). The natural immune cells may include but not limited to T-lymphocyte (T-cells), Natural Killer (NK) cells, Gamma Delta (γδ) T-cells, etc. In an exemplary embodiment, the ORF 100 is introduced in a natural T-cell via Clustered Regularly Interspaced Short Palindromic Repeats-CRISPR associated protein (CRISPR-Cas) based gene editing techniques. Additionally or alternatively, the ORF 100 is introduced in a natural T-cell using lentiviral vectors, Adeno-associated viruses (AAVs), etc. The vector including the ORF 100 can be selected from plasmid, cosmid, viral vector or phage. The plasmid may be a eukrayotic expression plasmid. The viral vector can be derived from a lentivirus, a retrovirus, an adenovirus, an adeno-associated virus, and/or a sendai virus. In an exemplary embodiment, a lentivirus (LV) is employed for introduction of the ORF 100 into the T-cell. Lentiviral (LV) vectors provide an effective means for modifying eukaryotic cells, stable transfer, and expression of gene in host cells with lower immunogenicity. LV vectors have larger gene accommodation capacity and can transduce proliferating as well as nonproliferating cells. Further, LV vectors-based gene delivery system is the most efficient method of transducing the hard-to-transfect T cells. In an exemplary embodiment, the clinical grade viral vectors were commercially obtained from Lentigen and Sirion-Biotech.

The ORF 100 may include one or more regions including one or more long terminal repeats (LTR) 110, one or more promoters, at least one hairpin loop structure, one or more genes, etc. The one or more promoters may include a first promoter 120 and a second promoter 140. The one or more genes may include but not limited to a plurality of CAR genes 150. The hairpin loop structure may include at least two short hairpin RNAs (shRNA) 130 sequences along with at least one microRNA30 (miR30) 130a sequence. Additionally or optionally, the ORF 100 may include one or more nucleotide sequences such as genes for fluorescent protein, c-myc, etc. In an exemplary embodiment, the hairpin loop structure regulates an amount of GMCSF cytokine during immunotherapy. A transcript (i.e., a messenger RNA) of the hairpin loop structure binds with a messenger RNA (mRNA) of a cytokine. In an exemplary embodiment, the transcript of the hairpin loop structure binds with the mRNA of GMCSF.

The LTR 110 may flank the ORF 100, i.e., the LTR 110 may be disposed at the 5′end and the 3′end of the ORF 100. In other words, the LTR 110 mays disposed upstream of the first promoter 120 and downstream of the CAR genes 150. In an exemplary embodiment, the LTR 110 at the 5′ end of the ORF 100 is encoded by SEQ ID NO. 1. The LTR 110 helps in integration of the ORF 100 in the native DNA of the T-cells while genetically manipulating the T-cells. Integration of the ORF 100 in the native DNA transforms the native T-cells into CAR T-cells (i.e., an exemplary engineered immune cell).

The transcription of the ORF 100 within the CAR-T cell may be controlled by the one or more promoters, i.e., the production of the transcripts of the one or more genes are controlled by the one or more promoters. The promoters may be disposed upstream of the one or more genes of the ORF 100. One or more RNA polymerase may bind to the promoters for transcription of the ORF 100. The RNA polymerase may include but not limited to eukaryotic RNA polymerase II (Pol II), eukaryotic RNA polymerase III (Pol III), etc. The RNA polymerase after binding with the promoters may partially or completely transcribe the ORF 100 to one or more messenger RNAs (mRNAs) (or transcripts). The mRNAs may be further processed to provide one or more CARs (protein structure) and shRNA 130 transcripts. Processing of mRNA may include at least one of splicing, translation of the mRNA to one or more amino acid sequences (i.e., a polypeptide) and post-translational modifications, etc. Post-translational modification may include but not limited to folding of the amino acid sequence (into a protein) and/or glycosylation, etc.

As shown in FIG. 2, the first promoter 120 may be disposed upstream of the shRNA 130 (and the hairpin loop structure(s)). The first promoter 120 may be selected from full-length cytomegalovirus (CMV) promoter (encoded by SEQ ID NO. 2), attenuated CMV promoter (encoded by SEQ ID NO. 3), human UG (hU6) promoter (encoded by SEQ ID NO. 4), mouse U6 (mU6) promoter, chicken 7SK (ch7SK) promoter, H1 promoter, SNORD promoter, etc.

The first promoter 120 transcribes the shRNA 130 to produce one or more short RNA transcripts having a hairpin loop structure. The shRNA 130 may be encoded by at least one of the SEQ ID NO. 5-SEQ ID NO. 24.

In an exemplary embodiment, as shown in FIG. 2, the ORF 100 is provided with two shRNA 130 sequences such that they complement each other and form a stem of the hairpin loop structure.

The transcript of the shRNA 130 may include a specific binding affinity towards one or more molecules. The CAR T-cell includes a plurality of GMCSF gene sequences (not shown) that produce pro-inflammatory cytokines (like GMCSF) which contributes to CRS. The shRNA 130 may inhibit cytokine production and/or cytokine signaling via RNA interference (RNAi). In an exemplary embodiment, the transcript of the shRNA 130 selectively binds with an mRNA corresponding to the cytokine (i.e., the GMCSF), to functionally inactivate the mRNA and/or degrade the mRNA.

In an exemplary embodiment, the shRNA 130 expressed by the CAR T-cell (i.e., the transcript of the shRNA 130) selectively binds with the GMCSF mRNA thereby regulating the GMCSF cytokine levels (via RNAi). Upon binding of the transcript of the shRNA 130 to the GMCSF mRNA, there is a decrease in the level (concentration) of the GMCSF molecules resulting in a reduced burden of CRS.

The miR30 130a is a short RNA sequence that acts as a regulatory element for the transcription of the shRNA 130 sequences, i.e., the miR30 130a at least partially regulates expression of the shRNA 130 sequence. The miR30 130a may flank the shRNA 130 sequences and/or be disposed in between the shRNA 130 sequences. In an exemplary embodiment, miR30 130a is encoded by SEQ ID No. 25. In an exemplary embodiment, as shown in FIG. 2, the miR30 130a sequence forms the loop of the hairpin loop structure formed by the shRNA 130 sequences. Expression of the shRNA 130 may be regulated by the miR30 130a and/or the first promoter 120.

Although excess of GMCSF production (as at the time of CRS) is detrimental, a low level of GMCSF promotes improved anti-tumor activity of the CAR T-cells. The miR30 130a helps to maintain low levels of GMCSF by regulating the expression of the shRNA 130 sequences. Thus, the miR30 130a sequence helps to retain the beneficial antitumor activity of the GMCSF while mitigating its potential CRS-inducing risks.

In an exemplary embodiment, the expression of the miR30 130a sequences are regulated by the host cell (i.e., the CAR T-cell). Since expression of the shRNA 130 sequences and the miR30 130a sequences are both controlled by the first promoter 120, the host cell is able to regulate the expression of the shRNA 130 sequence along with the miR30 130a sequence. Regulation of the expression of the shRNA 130 sequence by the host cell prevents overexpression of the shRNA 130 sequence. The shRNA 130 along with the miR30 130a, together allows for tight regulation on the levels of GMCSF. Thus, the CAR T-cells of the present disclosure has more precise and sustained suppression of GMCSF production.

Further, proper balancing (regulation) of GMCSF expression has led to suppression of the exhaustion markers on the CAR T-cells of the present disclosure. The said suppression of the exhaustion markers promotes a less exhausted and more functional CAR T-cell population, enhancing their long-term antitumor activity and persistence. The said enhanced persistence is crucial for long-lasting antitumor effects, as CAR T-cells need to survive and function in the hostile tumor microenvironment. Thus, the CAR T-cell survival and persistence are enhanced while reducing the risk of cytokine-related toxicities.

The shRNA 130 and the miR30 130a facilitates better infiltration of CAR T-cells into the tumor site. Improved tumor penetration is essential for CAR T-cells to recognize and target cancer cells effectively.

In an exemplary embodiment, the combination of shRNA 130 and miR30 130a provides enhanced infiltration of CAR T-cells into the tumor microenvironment. Regulation of GMCSF helps to modulate the tumor microenvironment, as GMCSF contributes to the recruitment and activation of various immune cells, including neutrophils, macrophages, and dendritic cells. Additionally, the incorporation of miR30 130a further augments the GMCSF inhibition. The miR30 130a acts as a post-transcriptional regulator that fine-tunes gene expression. By suppressing the translation of GMCSF mRNA, miR30 130a adds an extra layer of control to ensure minimal GMCSF production. This dual approach of shRNA 130 and miR30 130a synergistically acts to curtail GMCSF levels, thereby mitigating the immune interference that might otherwise hinder CAR T-cell infiltration into the tumor site. Thus, shRNA 130 and the miR30 130a facilitates better infiltration of CAR T-cells into the tumor site. Improved tumor penetration is essential for CAR T-cells to recognize and target cancer cells effectively.

FIG. 2a depicts an ORF 100a of the present disclosure. The ORF 100a is structurally same as ORF 100 with addition of an optional poly-A-tail 160 sequence, i.e., the ORF 100a includes the one or more LTRs 110, the at least two shRNA 130 sequence along with at least one miR30 130a sequence, the first promoter 120, the second promoter 140, a plurality of CAR genes 150, the poly-A-tail 160 sequence, etc. At least one hairpin loop structure may be formed by at least two short hairpin RNAs (shRNA) 130 sequences along with at least one microRNA30 (miR30) 130a sequence. In an exemplary embodiment, the shRNA 130 sequences forms a stem of the hairpin loop structure and the miR30 130a sequence forms a loop of the hairpin loop structure. A recombinant nucleotide sequence molecule is at least encoded by the ORF 100a. In an exemplary embodiment, the hairpin loop structure regulates an amount of GMCSF cytokine during immunotherapy. A transcript (i.e., a messenger RNA) of the hairpin loop structure binds with a messenger RNA (mRNA) of a cytokine. In an exemplary embodiment, the transcript of the hairpin loop structure binds with the mRNA of GMCSF.

The poly-A-tail 160 sequence may be disposed downstream of the shRNA 130 sequences or the miR30 130a sequence. Expression of the poly-A-tail 160 sequence may be regulated by the first promoter 120. In an exemplary embodiment, as shown in FIG. 2a, the poly-A-tail 160 sequence is downstream of the hairpin loop structure formed by the shRNA 130 sequence and the miR30 130a sequence. A transcript of the poly-A-tail 160 sequence may at least include a plurality of adenosine nucleotides. In an exemplary embodiment, the poly-A-tail 160 sequence is from either Simian virus 40 (SV40) or bovine growth hormone (bGH). The SV40 poly-A-tail 160 sequence is encoded by SEQ ID NO. 26. The poly-A-tail 160 helps in mRNA stability by offering protection against enzymatic cleavage, which is a process that could lead to mRNA degradation. It further ensures that the intended regulatory effects of the GMCSF and miR30 130a components are maintained over time, without undue degradation or interference.

FIG. 2b depicts an ORF 100b of the present disclosure. The ORF 100b is structurally same as ORF 100 without the miR30 130a sequence, i.e., the ORF 100b includes the one or more LTRs 110, the at least one shRNA 130 sequence (forming a hairpin loop structure), the first promoter 120, the second promoter 140, a plurality of CAR genes 150, etc. A recombinant nucleotide sequence molecule is at least encoded by the ORF 100b. Expression of the shRNA 130 sequence in the ORF 100b, depicted in FIG. 2b, is under the control of the first promoter 120. In an exemplary embodiment, the hairpin loop structure regulates an amount of GMCSF cytokine during immunotherapy. A transcript (i.e., a messenger RNA) of the hairpin loop structure binds with a messenger RNA (mRNA) of a cytokine. In an exemplary embodiment, the transcript of the hairpin loop structure binds with the mRNA of GMCSF.

Now referring to FIG. 2, the second promoter 140 may be disposed upstream of the CAR genes 150. In other words, the CAR genes 150 may be disposed downstream of the second promoter 140. The second promoter 140 may be selected from Elongation Factor 1 (EF1) full length promoter (encoded by SEQ ID NO. 27), EF1 alpha core promoter (encoded by SEQ ID NO. 28), etc. Accordingly, expression of the CAR genes 150 may be regulated by the second promoter 140.

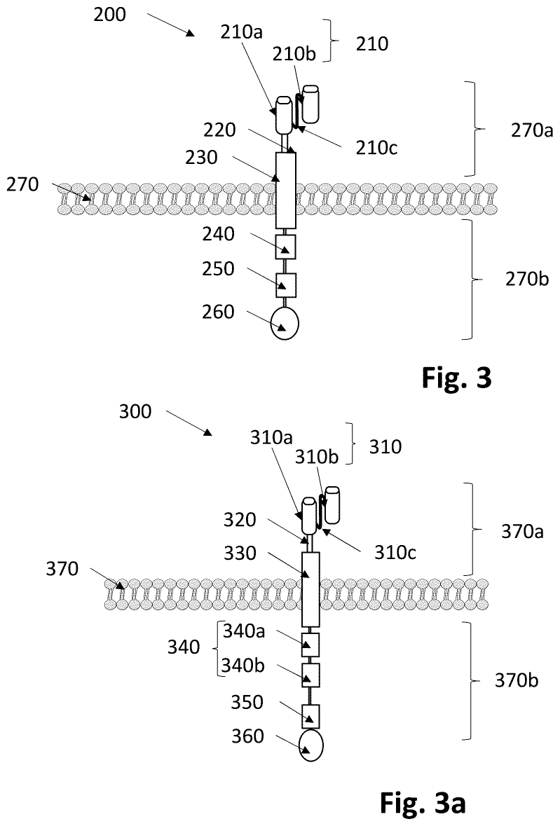

The CAR genes 150 include sequences encoding one or more domains of a CAR 200, as shown in FIG. 3. The one or more domains of the CAR 200 include but not limited to one or more single-chain variable fragment (scFV) domains 210, one or more hinge domains 220, one or more transmembrane domains 230, one or more co-stimulatory (CSTM) domains 240, one or more signaling domains 250, one or more safety switch (SS) domains 260, etc.

In an exemplary embodiment, as shown in FIG. 3, the CAR 200 includes one scFV domain 210, one hinge domain 220, one transmembrane domain 230, one CSTM domain 240, one signaling domain 250 and one SS domain 260. Additionally or optionally, the CAR 200 may include one or more peptide sequences disposed between the domains of the CAR 200 such as a signal peptide, a linker peptide, etc.

Like any antibody, the scFV domain 210 of the CAR 200 may include a light chain 210a and a heavy chain 210b. In an embodiment, the scFV domain 210 for the CD19 antigen (TAA) may be encoded by SEQ ID NO. 29 such that the scFV domain 210 includes a polypeptide sequence defined by SEQ ID NO. 30. In an alternate embodiment, the scFV domain 210 for the CD19 antigen (TAA) may be encoded by SEQ ID NO. 31 such that the scFV domain 210 includes a polypeptide sequence defined by SEQ ID NO. 32. The light chain 210a and the heavy chain 210b of the scFV domain 210 may be coupled to each other via a linker protein 210c. The scFV domain 210 may have a binding specificity (or affinity) towards one or more TAAs including but not limited to CD19, CD7, CD20, CD22, CD123, CD133, CD30, CD138, EGFR, EGFRvIII, FAP, MUC1, GD2, CEA, PSMA, HER2, NY-ESO-1, MAGEA-A3, hTERT, etc. In an exemplary embodiment, the CAR 200 includes one scFV domain 210 specific to CD19 antigens of B-lymphocytes (B-cells). Alternatively, the CAR 200 may include two or three scFV domains (not shown) for the same TAA or different TAAs.

The hinge domain 220 operationally couples the scFV domain 210 to the transmembrane domain 230. In an exemplary embodiment, the hinge domain 220 includes Cluster of Differentiation 8 (CD8) domain. The hinge domain 220 helps to provide flexibility to the scFV domain 210 with respect to the transmembrane domain 230. In an embodiment, the hinge domain 220 is encoded by SEQ ID NO. 33 such that the hinge domain 220 includes a polypeptide sequence defined by SEQ ID NO. 34.

The transmembrane domain 230 may be disposed across a bi-lipid cell membrane (or membrane) 270. The membrane 270 separates an extracellular region 270a from an intracellular region 270b. In an exemplary embodiment, the transmembrane domain 230 includes a CD8 domain encoded by SEQ ID NO. 35 such that the transmembrane domain 230 includes a polypeptide sequence defined by SEQ ID NO. 36. The transmembrane domain 230 helps to operationally couple the domains of the CAR 200 present in the extracellular region 270a to the domains of the CAR 200 present in the intracellular region 270b. In an exemplary embodiment, as shown in FIG. 3, the scFV domain 210 and the hinge domain 220 are the only domains present in the extracellular region 270a.

The CSTM domain 240 may be coupled to the transmembrane domain 230. The CSTM domain 240 may be selected from Cluster of Differentiation 28 (CD28), Inducible T-cell CO-Stimulator (ICOS), OX40, 4-1BB, DAP10, DAP12, 2B4, etc. The CD28 CSTM domain 240 may be encoded by SEQ ID NO. 37 such that the CD28 CSTM domain 240 includes a polypeptide sequence defined by SEQ ID NO. 38. The ICOS CSTM domain 240 may be encoded by SEQ ID NO. 39 such that the ICOS CSTM domain 240 includes a polypeptide sequence defined by SEQ ID NO. 40. The OX40 CSTM domain 240 may be encoded by SEQ ID NO. 41 such that the OX40 CSTM domain 240 includes a polypeptide sequence defined by SEQ ID NO. 42. The 4-1BB CSTM domain 240 may be encoded by SEQ ID NO. 43 such that the 4-1BB CSTM domain 240 includes a polypeptide sequence defined by SEQ ID NO. 44. The DAP10 CSTM domain 240 may be encoded by SEQ ID NO. 45 such that the DAP10 CSTM domain 240 includes a polypeptide sequence defined by SEQ ID NO. 46. The DAP12 CSTM domain 240 may be encoded by SEQ ID NO. 47 such that the DAP12 CSTM domain 240 includes a polypeptide sequence defined by SEQ ID NO. 48. The 2B4 CSTM domain 240 may be encoded by SEQ ID NO. 49 such that the 2B4 CSTM domain 240 includes a polypeptide sequence defined by SEQ ID NO. 50. In an embodiment, the CSTM domain 240 includes 4-IBB. The CSTM domain 240 helps to enhance cell-mediated immune response.

In an exemplary embodiment, the CAR T-cells are activated by subjecting the CAR T-cells to co-stimulatory molecules (for example, CD3 and/or CD28). The co-stimulatory molecules stimulate the CSTM domain 240, which in turn leads to activation, proliferation, and/or differentiation of the CAR T-cells into effector T cells (thus enabling the CAR T-cells to express the CAR construct). CD3/CD28 activation mimics the signals T cells receive during natural antigen recognition, thus, providing a safe and effective way to synthetically activate the CAR T-cells in vitro as well as in vivo. The CD3/CD28-activated CAR T-cells (or effector T cells) are then used to target and kill cancerous cells.

The signaling domain 250 may be coupled to the CSTM domain 240. In an exemplary embodiment, the signaling domain 250 is a Cluster of differentiation 3 zeta (CD35) domain encoded by SEQ ID NO. 51 such that the CD35 signaling domain 250 includes a polypeptide sequence defined by SEQ ID NO. 52. The signaling domain 250 helps to transmit an activation signal to the CAR T-cell after a TAA binds with the scFV domain 210.

The SS domain 260 may be coupled to the signaling domain 250. The SS domain 260 may be selected from iCaspase 9, EGFRt, RQR8, etc. In an exemplary embodiment, the SS domain 260 includes iCaspase 9 encoded by SEQ ID NO. 53 such that the iCaspase 9 SS domain 260 includes a polypeptide sequence defined by SEQ ID NO. 54. In another exemplary embodiment, the SS domain 260 includes RQR8 encoded by SEQ ID NO. 55 such that the RQR8 SS domain 260 includes a polypeptide sequence defined by SEQ ID NO. 56. The SS domain 260 may control the cell proliferation of the CAR T-cell thereby preventing transformation of the CAR T-cells into cancerous cells. In other words, in the presence of predefined inducers for iCaspase 9, EGFRt, and/or ROR8, the SS domain 260 allows selective depletion of CAR T-cells.

In an exemplary embodiment, the inducible caspase 9 (iCaspase9) SS domain 260 encodes a caspase recruitment domain (CARD; GenBank NM001 229) linked to 2 12-kDa human FK506 binding proteins (FKBP12; GenBank AH002 818) that contain an F36V mutation. Administration of a chemical inducer of dimerization (CID) results in the dimerization of the inducible caspase 9 molecules, leading to their activation. The caspase 9 dimer will subsequently activate downstream effector caspases, such as caspase 3, and ultimately induce cell apoptosis.

In another exemplary embodiment, the RQR8 SS domain 260 encodes a multi-epitope molecule harboring a CD34 epitope and two CD20 mimotopes. The CD20 mimotopes when bound to the FDA-approved CD20 antibody rituximab, the host cell undergoes cell apoptosis

FIG. 3a depicts another embodiment of a CAR 300. Similar to the CAR 200, the CAR 300 includes one or more scFV domains 310, one or more hinge domains 320, one or more transmembrane domains 330, one or more CSTM domains 340, one or more signaling domains 350, one or more SS domains 360, etc. The transmembrane domain 330 may be disposed across a membrane 370 such that the scFV domain 310 and the hinge domain 320 are disposed outside the CAR T-cell i.e., in an extracellular region 370a. The remaining domains of the CAR 300 may be disposed within the CAR T-cell i.e., in an intracellular region 370b.

In an exemplary embodiment, as shown in FIG. 3a, the CAR 300 includes one scFV domain 310, one hinge domain 320, one transmembrane domain 330, a first CSTM domain 340a and a second CSTM domain 340b, one signaling domain 350, and one SS domain 360.

Like the scFV domain 210 of the CAR 200, the scFV domain 310 of the CAR 300 may include a light chain 310a and a heavy chain 310b. The light chain 310a and the heavy chain 310b of the scFV domain 310 may be coupled to each other via a linker protein 310c.

Like CSTM domain 240 of the CAR 200, the first CSTM domain 340a of the CAR 300 may be selected from CD28, ICOS and/or OX40. The second CSTM domain 340b may be coupled to the first CSTM domain 340a. The second CSTM domain 340b may be selected from 4-IBB, DAP10, DAP12 and/or 2B4.

In an exemplary embodiment, the first CSTM domain 340a and the second CSTM domain 340b are CD28 and 4-IBB respectively. Compared to the single CSTM domain 240 in CAR 200, the first CSTM domain 340a and the second CSTM domain 340b of CAR 300 help to enhance the persistence of the CAR T-cells.

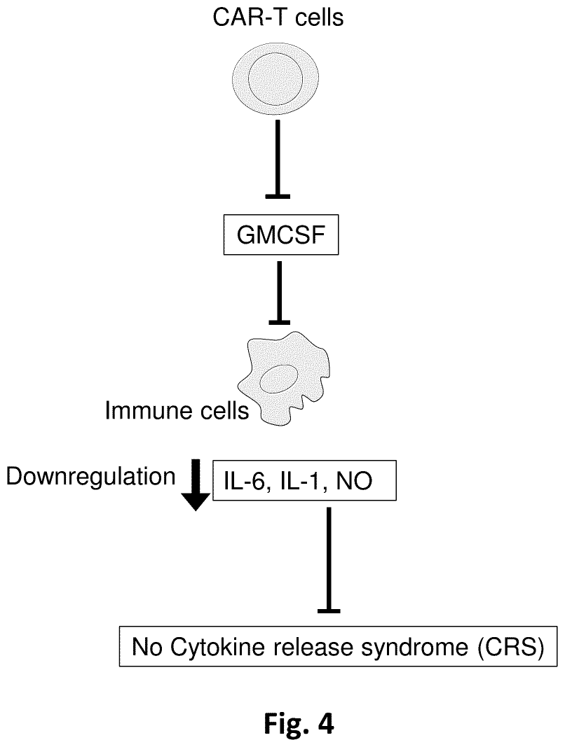

The above-described CAR T-cell produces negligible amount of cytokines like GMCSF as shown in FIG. 4. Therefore, the said CAR T-cell does not recruit any pro-inflammatory cells thereby down-regulating pro-inflammatory cytokines. Attenuation in levels of pro-inflammatory cytokines mitigate CRS.

Although, the shRNA 130 and miR30 130a are described with the examples of CAR 200/300 in the present disclosure, the shRNA 130 and miR30 130a may be used with any functionally equivalent CAR construct and the same is within the scope of the teachings of the present disclosure.

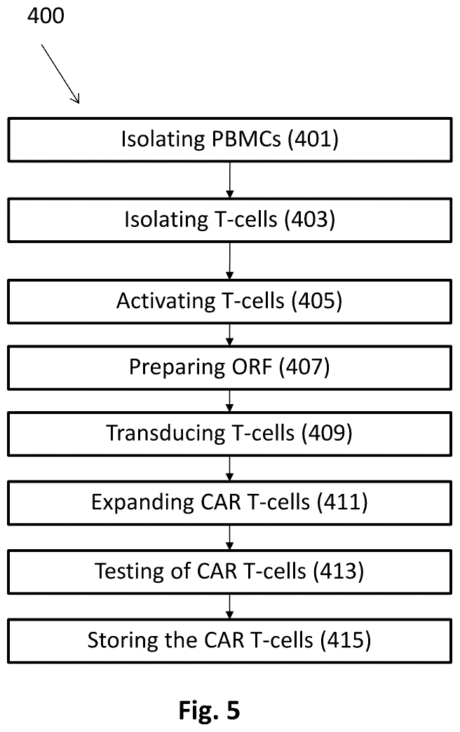

The CAR T-cells of the present invention may be prepared for each cancer patient by following a method 400 depicted in FIG. 5. Alternatively, the CAR T-cells may be prepared from an allogenic T-cell line for all cancer patients.

The method 400 begins at step 401 by isolating Peripheral Blood Mononuclear Cells (PBMCs) by either a ficoll-based method or an automated PBMC isolation instrument (as in case of Leukapheresis). In an exemplary embodiment, the PBMCs are isolated by drawing blood from a cancer patient requiring CAR T-cell based immunotherapy.

At step 403, a plurality of T-cells may be isolated from the PBMCs. The isolated T-cell population may include a plurality of CD4+ and CD8+ T-cells. The isolated T-cell population may include at least 40% CD4+ T-cells. In an exemplary embodiment, the isolated T-cell population include 40% CD4+ T-cells and 60% CD8+ T-cells.

At an optional step 405, the T-cells are activated by subjecting the T-cells to co-stimulatory molecules (for example, CD3 and/or CD28). In an exemplary embodiment, the co-stimulatory molecules help the T-cells to become competent to receive the ORF 100 via the lentiviral vectors (described below).

At step 407, the ORF 100/100a/100b of the present invention is prepared for delivery within the isolated T-cell population. The ORF 100/100a/100b may be ligated and replicated in a predefined vector (or plasmid) including but not limited to pHR, pTRPE lentiviral vectors, etc. In an exemplary embodiment, the ORF 100/100a/100b is replicated and packaged within lentiviral particles.

At step 409, the ORF 100/100a/100b (along with the vector) is delivered within the isolated T-cell population via a pre-defined gene delivery method. The pre-defined gene delivery method may be one of transformation, transfection, or transduction. In an exemplary embodiment, the isolated T-cell population is transduced by Lentiviral based gene delivery system such as the third-generation self-inactivating (SIN) lentiviral system. In an exemplary embodiment of the present disclosure, the ORF 100/100a/100b is delivered to the T-cells via third generation lentiviral particles, i.e., the T-cells are transduced with third generation lentivirus. The resultant CAR T-cells target and bind to specific cancer cells, thereby allowing the CAR T-cells to recognize and attack the CD19 expressing B cells (i.e., the cancerous cells).

At step 411, the CAR T-cells (or engineered immune cells) are expanded in a pre-defined nutrient medium for a pre-defined time to increase the number of CAR T-cells. In an exemplary embodiment, the CAR T-cells were expanded for 7 to 9 days to increase the number of CAR T-cells by 20-40 times. The engineered immune cells suspended in the pre-defined nutrient medium may define a composition.

In an exemplary embodiment, the nutrient medium includes AIM V media with or without antibiotic, human serum albumin, phenol red and L glutamine. In an alternate embodiment, the nutrient medium includes CTS AIM V media without antibiotics, phenol red and serum. The nutrient medium may be periodically replaced with fresh nutrient medium after a pre-defined time. In an exemplary embodiment, the nutrient medium is replaced every 48 hours.

At step 413, the transduced T-cells expressing the CAR 200, 300 are selected and isolated by subjecting the transduced T-cells to in vitro and/or in vivo testing techniques. Additionally or alternatively, the transduced T-cells expressing the CAR 200, 300 are selected by immunoassay-based selection or by cell sorting techniques.

At an optional step 415, the selected transduced T-cells expressing the CAR 200, 300 (i.e., the CAR T-cells are refrigerated for storage and transportation The frozen CAR T-cells are thawed prior to infusion into the patient.

Alternatively, the selected T-cells (i.e., CAR T-cells) may be directly infused into the patient as decided by the physician.

Although the method 400 is described with an exemplary sequence of steps, the steps of the method 400 may be rearranged while practicing the method 400 and the same is within the scope of the teachings of present disclosure.

Sequences sharing at least 95% identity with the sequences disclosed in the present disclosure are within the scope of the teachings of the present disclosure.

The present disclosure is further described by reference to the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples, but rather, should be construed to encompass any and all variations which become evident as a result of the teaching provided herein.

### Example 1: CAR T-Cells Preparation Methodology (Present Invention)

The CAR T-cells were prepared as described below. Unless specifically mentioned, the CAR T-cells were cultured in a humidified incubator at 37° C., 5% CO₂ as per the experimental requirements. Lentiviral packaging HEK293T cells obtained from American Type Culture Collection (ATCC) were seeded at approximately 3.8×10⁶ cells per plate in Dulbecco's Modified Eagle Medium (DMEM) complete in 10 cm tissue culture plates. The tissue culture plates including the HEK293T cells were then incubated at 37° C., 5% CO₂ for about 24 hours. After about 24 hours, the old medium was aspirated out and replaced with 10 mL fresh DMEM complete and 25 μM cloroquine diphosphate. The tissue culture plates including the HEK293T cells were further incubated at 37° C., 5% CO₂ for about 6 hours.

A mixture of lenti-plasmids and CAR plasmid (including the ORF 100a of the present disclosure) were inserted into the HEK293T cells using Polyethylenimine (PEI, a non-viral vector) as the transfection reagent. The HEK293T cells were incubated at 37° C., 5% CO₂ for about another 36 hours before harvesting the medium containing the viral particles at different intervals, up to 72 hours. The harvested medium was centrifuged at 1000 rpm for 5 minutes to pellet any remaining HEK293T cells. The supernatant was then filtered using a 0.45 μm PES filter. The filtrate (supernatant) containing the viral particles were collected and refrigerated to avoid loss of titer. The viral particles had the lenti-plasmid integrated with the CAR plasmid (including the ORF 100a of the present disclosure).

10 million PBMCs were collected per 20 ml of the patient's blood through a process called Leukapheresis. T-cells were then isolated from the collected PBMCs using RosetteSe Human CD4+ T Cell Enrichment assay and RosetteSe Human CD8+ T Cell Enrichment assay. The isolated T-cell population had 40% CD4+ T-cells and 60% CD8+ T-cells. The isolated T cells were cryopreserved in RPMI-1640 with 20% human AB serum and 10% DMSO.

The isolated T cells are cultured in human T cell medium consisting of X-VIVO 15 (Lonza), 5% Human AB serum, and 10 mM neutralized N-acetyl L-Cysteine (Sigma-Aldrich) in a humidified CO2 incubator at 37° C. Further, IL-2 were used for cell proliferation and used at 30 units/mL IL-2.

After 24 hours in culture, the isolated T-cells were stimulated (activated) with Human T-Activator CD3/CD28 Dynabeads (Life Technologies) at a 1:3 cell:bead ratio.

At 48 hours, the primary T cells were exposed to the supernatant (containing the viral particles) for transduction. At day 4 after T cell stimulation, the Dynabeads were removed, and the T cells were expanded until day 9. The T-cells were sorted with FACs ARIA II. Thus, the T-cells exhibiting basal CAR expression (i.e., the CAR T-cells) were isolated.

The isolated CAR T-cells were expanded in the nutrient medium including CTS AIM V media without antibiotics, phenol red and serum at 37° C., 5% CO2. The nutrient medium was replaced every 48 hours with fresh nutrient medium. It was observed that after expanding the CAR T-cells for about 7-9 days, the number of CAR T-cells increased by 20-40 times. The CAR T-cells were frozen for storage and transportation.

### Example 2: Infusion of CAR T-Cells (Present Invention)

The CAR T cells (as obtained in Example 1) were thawed. About 1×10⁶ CAR T-cells per kilogram weight of a patient's weight were then intravenously infused into the patient in an outpatient setting. The CAR T-cells quickly migrated to the bone marrow and lymphoid tissues. After migration, the CAR T-cells had selectively bound to the cancer cells expressing CD19. The binding between the CAR T-cell and the cancerous cell triggered activation and proliferation of the CAR T-cells, after which the cancer cells were killed via mechanisms such as cytokine release and cytotoxicity.

The CAR T-cells persisted in the patient and continued to attack CD19-expressing cancerous cells, providing long-term therapeutic effect to the patient. It was observed that the CAR T-cells of the present disclosure provided response rates of up to 90% in patients with acute lymphoblastic leukemia and up to 50-60% in patients with non-Hodgkin lymphoma. The CAR T-cells specifically targeted cancer cells expressing CD19, reducing the risk of harm to healthy cells and reducing the risk of false-positive interactions. Unlike traditional chemotherapy and radiation therapy, CAR T cell therapy has minimal toxicity to healthy cells.

### Example 3: Evaluation of the CAR T-Cells of the Present Disclosure

Thirty transgenic mice were used in the study. The transgenic mice were four weeks old pathogen free NOD CRISPR Prkdc II2r Gamma (NCG) mice. These mice were purchased from Charles River laboratories, and were housed in a transgenic animal facility. The mice were housed in individually ventilated cages in a barrier system, which were designed to keep the animals in a clean and controlled environment. The conditions of the housing were carefully controlled to ensure the well-being of the mice, including a temperature range of 20-26 degrees Celsius, a relative humidity range of 40-65%, a 12-hour light-dark cycle, and an air exchange rate of over 50 times per hour. The mice were provided with ad libitum access to a certified rodent diet, which is specially formulated to meet their nutritional needs and regular body weight was monitored. They were also given sterilized water ad-libitum via water bottles. Before the study was conducted, the mice were quarantined for 15 days to ensure that they were free from any potential pathogens that could affect the results of the experiment. This quarantine period was as per a standard practice prescribed in animal research to minimize the risk of contamination and ensure the validity of the study. At the beginning of experiment, it was ensured that they are healthy, well-cared-for, and free from potential contaminants.

Raji cells (Burkitt's lymphoma) were obtained from American Type Culture Collection (ATCC) from which the Raji-luciferase (Luc) cells were generated. These cells were generated by stably infusing Raji cells with firefly luciferase as a reporter. The Raji-Luc cells were then recovered and grown using RPMI culture medium, which contained 10% fetal bovine serum (FBS) and 1% penicillin-streptomycin solution. The cells were cultured in an incubator set at 37 degrees Celsius and 5% CO2, which was the optimal environment for cell growth. Before use, the cells were adjusted to a concentration of 1 million cells per 200 micro liters using 0.9% NaCl or PBS.

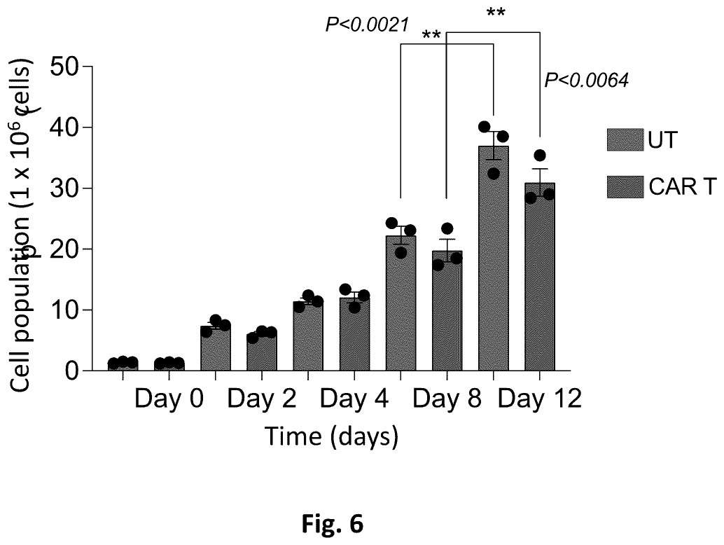

The CAR T-cells (CART) of the present disclosure (as obtained in example 1) and untransduced T-cells (UT) were cultured in presence of IL7 and IL1-5 for indicated time points (as shown in FIG. 6) and the cell viability was determined by counting the live cells. The data was plotted as the rate of cell proliferation indicating cell expansion and survival. Data is shown as Mean±SEM of three independent experiments calculated by non-parametric t test between UT (P<0.0021) or CART (P<0.0064) cells at day 8 and day 12, suggesting significant proliferation of the CAR T-cells.

The transgenic mice were divided into five groups, namely, Group 1, Group 2, Group 3, Group 4, Group 5. The transgenic mice in Group 1 (Sham) were the untreated control. The transgenic mice in Group 2 (Raji) were injected (by tail vein injection) with 1×10⁶ Raji-Luc cells on day 0. The transgenic mice in Group 3 (Raji+UT) were injected with (by tail vein injection) 1×10⁶ Raji-Luc cells on day 0 followed by the untransduced T-cells on day 5. The transgenic mice in Group 4 (Raji+CART^{con}) were injected with 1×10⁶ Raji-Luc cells on day 0 followed by infusion of conventional CAR T-cells on day 5. The conventional CAR T-cells had CAR construct with only one co-stimulatory domains, i.e., 4-1BB. And, the transgenic mice in Group 5 (Raji+CARTshGMCSF-miR30) were injected with 1×10⁶ Raji-Luc cells on day 0 followed by infusion of 1×10⁷ CAR T-cells of the present disclosure on day 5. The CAR T-cells of the present disclosure had CAR construct 300 with two CSTM domains 340, i.e., CD28 and 4-1BB.

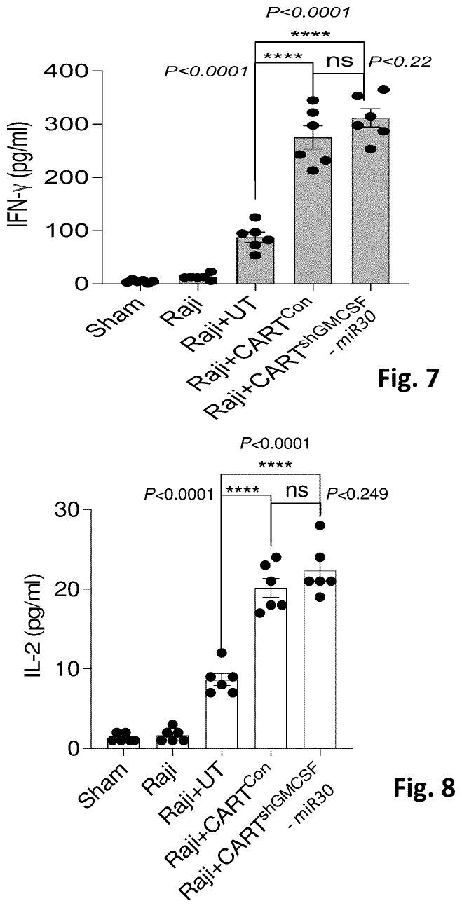

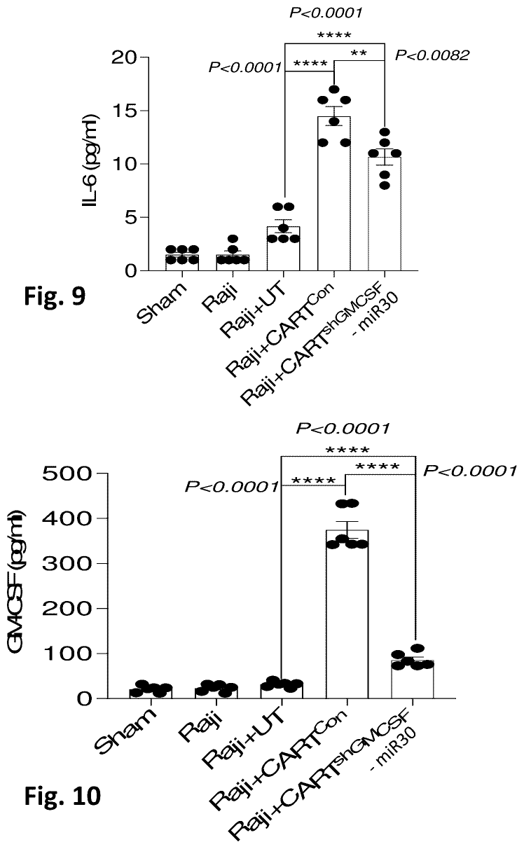

Blood samples were taken from the animals to measure the levels of various human cytokines, such as IFN-γ (as shown in FIG. 7), IL-2 (as shown in FIG. 8), IL-6 (as shown in FIG. 9), and GM-CSF (as shown in FIG. 10), which indicates the inflammatory response.

The analysis of the measured cytokine levels showed that the levels of IFN-γ, IL-2, and IL-6 were increased in the groups given CAR T-cells, with IFN-γ and IL-2 being present at higher levels in both groups given CART^{Con} and CARTshGMCSF-miR30 (See FIG. 7 and FIG. 8 respectively). However, IL-6 was found to be significantly lower in the CARTshGMCSF-miR30 group compared to the CART^{Con} group (** indicates P<0.0082) (See FIG. 7). Additionally, the group administered CARTshGMCSF-miR30 had a significant decrease in GM-CSF levels compared to the CART^{Con} group (** indicates P<0.001), indicating a lower burden of cytokine release syndrome (See FIG. 8).

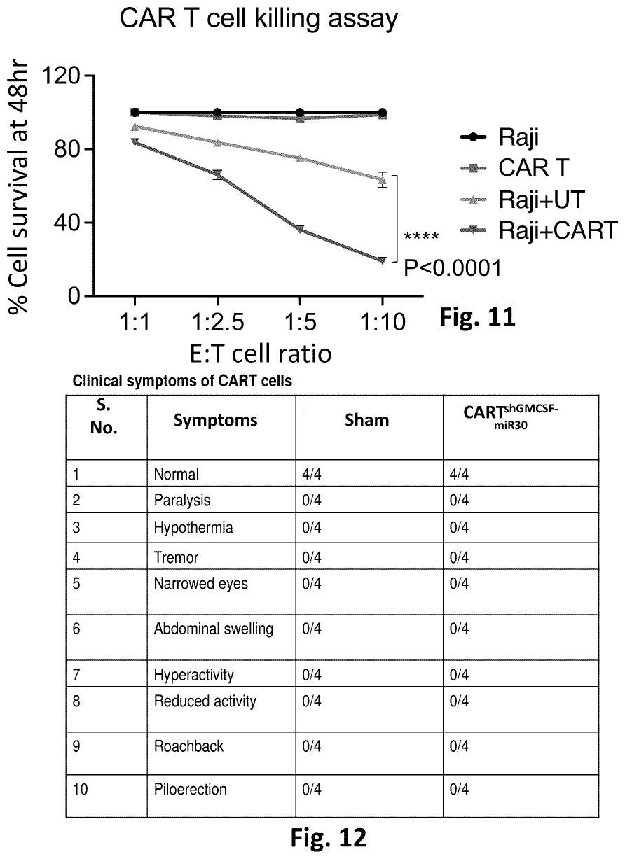

Further, as shown in FIG. 11, the anti-tumor activity was evaluated through Bioluminescence. The CAR T-cells (CART) and untransduced T-cells (UT) were co-cultured with Raji-luciferase expressing tumor cells for 48 hours at various effector to target (E:T) cell ratios, the target cells being the Raji-Luc cells (Raji). The anti-tumor activity was then measured by incubating the cells with D-luciferin and using a multi-mode plate reader (Perkin Elmer) to take readings. The results are presented as the percentage of surviving Raji-Luc cells (and in case of the sample without any Raji-Luc cells, the percentage survival of CART cells is represented), after normalization with the background signal. Data is shown as Mean±SEM of three independent experiments with **** indicating P<0.0001 calculated by non-parametric t test between untransduced T cells (UT) after co-cultured with Raji cells (Raji+UT) and CART cells with Raji cells (Raji+ CART).

With respect to FIG. 12, in vivo oncogenic potential or tumorigenic studies of CAR T cells was conducted where the mice were treated with only the CAR T-cells (1×10⁷ cells per mice) of the present disclosure (CARTshGMCSF-miR30) for a period of 120 days. The said mice were assessed with respect to untreated mice (Sham). At the end of 120 days, the mice were sacrificed, and histopathology examination of the tissues was conducted to evaluate tumorigenesis. The results of the study indicated that there were no tumors found in any of the organs studied. Additionally, blood profiling was done, and no CAR T-cells were detected. Furthermore, clinical symptoms of the animals were evaluated, and no signs of the tumor development were observed. These findings confirm that the CAR T-cells of the present disclosure do not induce in vivo oncogenesis and can be considered safe for use in humans. It is important to note that CAR T-cells of the present disclosure further includes a safety switch, which can eliminate the CAR T-cells from the system in case any tumorigenesis is observed in the patients. This further increases the safety of the CAR T-cells of the present disclosure. Therefore, the results presented eliminate the in vivo oncogenic potential or tumorigenic studies of CAR T-cells of the present disclosure and provide strong evidence for the safety of this cellular therapy.

The conclusion of this study is that the CAR T-cells of the present disclosure are effective in treating cancer in mice. The study also revealed that mice treated with CARTshGMCSF-miR30 showed better survival, faster tumor clearance, and highly reduced levels of pro-inflammatory GM-CSF and IL-6. This suggests that CARTshGMCSF-miR30 has less cytokine release as compared to the conventional CART cell (CART^{Con}). Additionally, there were no signs of tissue toxicity or any clinical symptoms in the CART treated mice, indicating that CART cells are safe and do not pose any toxicity issues.

### Example 4: GMCSF Knockdown (Present Invention)

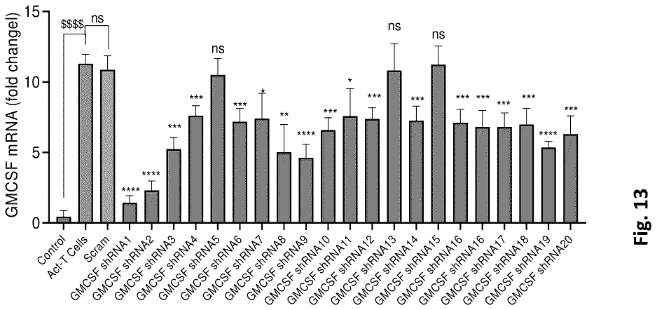

The 20 shRNA sequences along with the miR30 sequence as described in the present disclosure were evaluated for their effect on regulation of the GMCSF levels. The screening was done in T cells. The bar graph, as shown in FIG. 13, depicts GMCSF expression in T cells which are activated (Act-T cells) for 24 hours to induce GMCSF expression or unactuated (control). Non-targeting shRNA (Scram) and various GMCSF targeting shRNAs (having SEQ ID NO. 5 to 24) along with the miR30 (having SEQ ID NO. 25) were evaluated in activated T cells for their effect in knocking down of the GMCSF gene. T cells were transfected with respective shRNA-miR30 and mRNA was extracted after 24 hours of transfection. The assay was performed by RT-qPCR using GMCSF mRNA specific primers with SEQ ID NO. 57 and 58. The statistical analysis was done using the non-parametric t test indicating the P<0.0001 between control and Act-T cells, and ****P<0.0001; ***P<0.001; **P<0.01; *P<0.05; between GMCSF shRNA-miR30 and Scram expressing T cells. ns indicates non-significant (See FIG. 11). It was observed that the shRNA-miR30 sequences of the present disclosure significantly reduce the expression of the GMCSF gene via RNAi.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

1.-73. (canceled)

74. A recombinant nucleic acid molecule comprising:
i. at least one hairpin loop structure formed by one or more short hairpin RNA sequences, the at least one hairpin loop structure regulating an amount of Granulocyte monocyte colony stimulating factor (GMCSF) cytokine during immunotherapy, the short hairpin RNA is encoded by at least one of SEQ ID NO. 5-SEQ ID NO. 24 or a nucleotide sequence with at least 95% identity with at least one of the SEQ ID NO. 5-SEQ ID NO. 24; and
ii. a first promoter upstream of the at least one hairpin loop structure.

75. The recombinant nucleic acid molecule according to claim 74, wherein the first promoter is selected from a full-length cytomegalovirus (CMV) promoter, an attenuated CMV promoter, a human UG (hU6) promoter, a mouse U6 (mU6) promoter, a chicken 7SK (ch7SK) promoter, a H1 promoter, or a SNORD promoter.

76. The recombinant nucleic acid molecule according to claim 74, further comprising at least one microRNA30 (130a) sequence forming a loop of the at least one hairpin loop structure or flanking the at least one hairpin loop structure.

77. The recombinant nucleic acid molecule according to claim 74, further comprising a poly-A-tail sequence downstream of the hairpin loop structure, the poly-A-tail sequence being selected from either a Simian virus 40 (SV40) or a bovine growth hormone beta globulin (bGH).

78. The recombinant nucleic acid molecule according to claim 74, further comprising one or more chimeric antigen receptor genes downstream of a second promoter, the second promoter is selected from an Elongation Factor 1 (EF1) full-length promoter, or a EF1 alpha core promoter.

79. The recombinant nucleic acid molecule according to claim 74, further comprising one or more chimeric antigen receptor genes, the chimeric antigen receptor gene encodes one or more chimeric antigen receptors comprising at least one or more of:
i. at least one single-chain variable fragment domain encoded by at least one of SEQ ID NO. 30 or 32,
ii. at least one hinge domain coupled to the single-chain variable fragment domain, the hinge domain at least partially comprising a Cluster of Differentiation 8 (CD8),
iii. at least one co-stimulatory domain selected from at least one of a Cluster of Differentiation 28 (CD28), an Inducible T-cell CO-Stimulator (ICOS), a OX40, a 4-1BB, a DAP10, a DAP12, or a 2B4,
iv. at least one signaling domain coupled to the co-stimulatory domain, the signaling domain at least partially comprises a Cluster of differentiation 3 zeta (CD35),
V. at least one safety switch domain coupled to the signaling domain, the safety switch domain is selected from an inducible caspase 9 (iCaspase 9), a truncated Epidermal growth factor receptor (EGFRt), or a RQR8, and
vi. at least one transmembrane domain operationally couples the hinge domain to the co-stimulatory domain, the transmembrane domain at least partially comprising the Cluster of Differentiation 8 (CD8).

80. The recombinant nucleic acid molecule according to claim 74, further comprising one or more long terminal repeats upstream of the first promoter and downstream of one or more chimeric antigen receptor genes.

81. A transcript of the recombinant nucleic acid molecule according to claim 74, the transcript comprising a messenger RNA of the hairpin loop structure transcribed from the recombinant nucleic acid molecule.

82. A vector comprising the recombinant nucleic acid molecule according to claim 74, wherein the vector is selected from a plasmid, a cosmid, a viral vector or a phage, the viral vector is derived from at least one of a lentivirus, a retrovirus, an adenovirus, an adeno-associated virus, or a sendai virus.

83. An engineered immune cell comprising the recombinant nucleic acid molecule according to claim 74.

84. A composition comprising the engineered immune cell according to claim 83, the engineered immune cell is suspended in a nutrient medium, the nutrient medium comprises one of:
i. AIM V media with or without antibiotic, human serum albumin, phenol red and L glutamine; or
ii. CTS AIM V media without antibiotics, phenol red and serum.

85. An open reading frame comprising:
i. at least one hairpin loop structure formed by one or more short hairpin RNA sequences, the at least one hairpin loop structure regulating an amount of Granulocyte monocyte colony stimulating factor (GMCSF) cytokine during immunotherapy, the short hairpin RNA is encoded by at least one of SEQ ID NO. 5-SEQ ID NO. 24 or a nucleotide sequence with at least 95% identity with at least one of the SEQ ID NO. 5-SEQ ID NO. 24; and
ii. a first promoter upstream of the at least one hairpin loop structure.

86. The open reading frame according to claim 85, wherein the first promoter is selected from a full-length cytomegalovirus (CMV) promoter, an attenuated CMV promoter, a human UG (hU6) promoter, a mouse U6 (mU6) promoter, a chicken 7SK (ch7SK) promoter, a H1 promoter, or a SNORD promoter.

87. The open reading frame according to claim 85, further comprising at least one microRNA30 (130a) sequence forming a loop of the at least one hairpin loop structure or flanking the at least one hairpin loop structure.

88. The open reading frame according to claim 85, further comprising a poly-A-tail sequence downstream of the hairpin loop structure, the poly-A-tail sequence is selected from a Simian virus 40 (SV40) or a bovine growth hormone beta globulin (bGH).

89. The open reading frame according to claim 85, further comprising one or more chimeric antigen receptor genes downstream of a second promoter, the second promoter is selected from an Elongation Factor 1 (EF1) full-length promoter, or a EF1 alpha core promoter.

90. The open reading frame according to claim 85, further comprising one or more chimeric antigen receptor genes, the chimeric antigen receptor gene encodes one or more chimeric antigen receptors comprising at least one or more of:
i. at least one single-chain variable fragment domain encoded by at least one of SEQ ID NO. 30 or 32,
ii. at least one hinge domain coupled to the single-chain variable fragment domain, the hinge domain at least partially comprising a Cluster of Differentiation 8 (CD8),
iii. at least one co-stimulatory domain selected from at least one of a Cluster of Differentiation 28 (CD28), an Inducible T-cell CO-Stimulator (ICOS), a OX40, a 4-1BB, a DAP10, a DAP12, or a 2B4,
iv. at least one signaling domain coupled to the co-stimulatory domain, the signaling domain at least partially comprises a Cluster of differentiation 3 zeta (CD35),
V. at least one safety switch domain coupled to the signaling domain, the safety switch domain is selected from an inducible caspase 9 (iCaspase 9), a truncated Epidermal growth factor receptor (EGFRt), or a RQR8, and
vi. at least one transmembrane domain operationally couples the hinge domain to the co-stimulatory domain, the transmembrane domain at least partially comprising the Cluster of Differentiation 8 (CD8).

91. The open reading frame according to claim 85, further comprising one or more long terminal repeats upstream of the first promoter and downstream of one or more chimeric antigen receptor genes.

92. A transcript of the open reading frame according to claim 85, the transcript comprising a messenger RNA of the hairpin loop structure transcribed from the open reading frame.

93. A vector comprising the open reading frame according to claim 85, wherein the vector is selected from a plasmid, a cosmid, a viral vector or a phage, the viral vector is derived from at least one of a lentivirus, a retrovirus, an adenovirus, an adeno-associated virus, or a sendai virus.

94. An engineered immune cell comprising the open reading frame according to claim 85.

95. A composition comprising the engineered immune cell according to claim 94, the engineered immune cell suspended in a nutrient medium, the nutrient medium comprises one of:
i. AIM V media with or without antibiotic, human serum albumin, phenol red and L glutamine; or
ii. CTS AIM V media without antibiotics, phenol red and serum.

96. A recombinant nucleic acid molecule encoded at least by an open reading frame to lower a cytokine storm during immunotherapy, the open reading frame as claimed in claim 85.
